(19) United States
(12) Patent Application Publication
(10) Pub. No.: US 2012/0283687 A1
(43) Pub. Date: Nov. 8, 2012

(54) DIAPER COVER

(76) Inventors: Hirofumi Miyake, Mima-gun (JP); Akiko Tatsukawa, Mima-gun (JP)

(21) Appl. No.: 13/520,402

(22) PCT Filed: Jan. 28, 2011
(86) PCT No.: PCT/JP2011/000486

§ 371 Date: Jul. 3, 2012

(30) Foreign Application Priority Data
Feb. 3, 2010 (JP) 2010-022443
Feb. 3, 2010 (JP) 2010-022444
Mar. 2, 2010 (JP) 2010-045755

(51) Int. Cl. A61F 13/72

(52) U.S. Cl. 604/396

(57) ABSTRACT

A diaper cover comprising a front part, a back part and an intermediate part located between the front part and the back part in a front-back direction, a right waist part and a left waist part extending from the back part in a width direction, and a right flap part and a left flap part extending from the front part in the width direction, wherein each of the right flap part and the left flap part is provided with a first attachment, the right flap part, the left flap part and the intermediate part are stretchable, and the first attachment is attachable to a position on a dorsal side of a wearer in a state where the right flap part and the left flap part are stretched.

### TECHNICAL FIELD

The present invention relates to a diaper cover.

### BACKGROUND ART

Conventionally, there is known a diaper cover comprising a right waist part and a left waist part extending in a width direction from a back part of a front-back direction, and a right flap part and a left flap part extending in the width direction from the front part of the front-back direction. In such a diaper cover, the right waist part and the left waist part are fixed around a wearer, and the right flap part and the left flap part are fixed on the abdomen side of the wearer, whereby the diaper cover can be worn. For example, Patent Literature 1 discloses a diaper cover comprising a right waist part and a left waist part extending in a width direction from a back part of a front-back direction, and a right flap part and a left flap part extending in the width direction from the front part of the front-back direction, wherein the right waist part, the left waist part, the right flap part and the left flap part are stretchable.

### CITATION LIST

### Patent Literature

PTL 1: Japanese Patent Application Laid-Open Publication No. 2006-42863

### SUMMARY OF INVENTION

### Technical Problem

For elderly people who need to wear a diaper, it is often seen that their daily life is interfered due to the fact that their legs and back are weaken and going up and down stairs or walking on their own becomes difficult. In addition, when it becomes difficult to make their trunk upright due to weakening of muscles, it narrows the range of activities, which may cause a vicious cycle in terms of maintaining the physical motor function. As a muscle which controls standing posture and walking, a iliopsoas muscle is known which extends from about a groin to a dorsal side of a waist and a back, for example. However, in the diaper cover disclosed in the Patent Literature 1, it is difficult to suppress weakening of wearer's legs and back or strengthen wearer's legs and back.

The present invention has been achieved in view of the above circumstances, and an object of the present invention is to provide a diaper cover which can support an iliopsoas muscle of a wearer.

### Solution to Problem

A diaper cover of the present invention which solves the above problems comprises a front part, a back part and an intermediate part located between the front part and the back part in a front-back direction, a right waist part and a left waist part extending from the back part in a width direction, and a right flap part and a left flap part extending from the front part in the width direction, wherein each of the right flap part and the left flap part is provided with a first attachment, the right flap part, the left flap part and the intermediate part are stretchable, and the first attachment is attachable to a position on a dorsal side of a wearer in a state where the right flap part and the left flap part are stretched.

In the diaper cover of the present invention, since the right flap part, the left flap part and the intermediate part are stretchable, and the first attachment is attachable to a position on the dorsal side of a wearer in the state where the right flap part and the left flap part are stretched, tension is imparted to the diaper cover from about the groins of the wearer through about the inguinal regions on the abdomen side to the dorsal side of the waist. As a result, the iliopsoas muscle of the wearer is reinforced by the diaper cover. In the state where the wearer stands, the diaper cover of the present invention serves so as to keep the pelvis leaned forward and supports the iliopsoas muscle so as to assist the wearer to easily take a standing posture. When the wearer bends his or her upper body in the front-back direction and the right-left direction of the wearer, the diaper cover acts as a load with respect to the movement of the waist, and thus the iliopsoas muscle is developed. Therefore, even for elderly people who need to wear a diaper, weakening of legs and back is suppressed by wearing the diaper cover of the present invention.

The first attachment is preferably attachable to a position on the dorsal side of the pelvis of the wearer. When the first attachment is fixed to a position on the dorsal side of the pelvis of the wearer in the state where the right flap part and the left flap part are stretched, the portion of the pelvis from about the groins of the wearer to the dorsal side of the waist is certainly supported by the tension of the diaper cover, and thus the wearer easily keeps a standing posture.

The first attachment is preferably attachable to a position as far as 5% or more and 20% or less of a waist circumference length of the wearer away from a backbone of the wearer. When the first attachment is attached to such a position, the iliopsoas muscle is likely to be certainly supported by the diaper cover.

It is preferred that the stretch rate of each of the right flap part, the left flap part and the intermediate part in the front-back direction is higher than that in the width direction. When the stretch rate of each the right flap part, the left flap part, and the intermediate part in the front-back direction is higher than that in the width direction, the wearer is easily supported by the diaper cover at a portion from about the inguinal regions to the dorsal side of the waist, more effectively.

The stretch rates of the right flap part and the left flap part are preferably higher than that of the intermediate part. When the diaper cover is constituted in such a manner, the tension of the diaper cover becomes higher about the inguinal regions of the wearer than at the crotch of the wearer. Therefore, wearing feel of the diaper cover at the crotch improves, and the diaper cover also can effectively support the wearer from about the inguinal regions to the dorsal side of the waist. Furthermore, when the diaper cover is worn, a diaper such as an incontinent pad is easily disposed to the intermediate part without being twisted or bent.

It is preferred that the first attachment has a practically straight line shape having a width and the first attachment is provided such that the practically straight line shape extends forward in the front-back direction and inward in the width direction. By providing the first attachment in such a manner, the wearer easily pulls the right flap part and the left flap part upward while expanding the right flap part and the left flap part in the right-left direction of the wearer, whereby the tension is easily imparted to the diaper cover from the crotch of the wearer through about the inguinal regions on the abdomen side to the dorsal side of the wearer.

It is preferred that the right waist part and the left waist part are stretchable, one or both of the right waist part and the left waist part is provided with a second attachment, and the diaper cover is formed such that the right waist part and the left waist part cover iliac crests of the wearer and are joined together with the second attachment when the diaper cover is worn. When the right waist part and the left waist part are constituted in such a manner, both sides of the pelvis are covered with the right waist part and the left waist part to support the pelvis, and thus widening or distortion of the pelvis, which is a cause of low back pain, is easily corrected or prevented by the diaper cover.

The stretch rates of the right waist part and the left waist part are preferably higher than that of the back part. When the stretch rate of the back part is lower than that of each of the right waist part and the left waist part, the first attachments can be easily fixed to desired positions on the dorsal side of the wearer by attaching the first attachments of the right and left flap parts to the back part or its vicinity. As a result, the iliopsoas muscle of the wearer is likely to be surely reinforced by the diaper cover.

In the diaper cover of the present invention, it is preferred that both of the right waist part and the left waist part are provided with the second attachments, an end of one of the right waist part and the left waist part is located anterior to an end of the other of the right waist part and the left waist part in the front-back direction, whereby the right waist part and the left waist part are joinable together so as to be displaced each other in the front-back direction. Alternatively, in the diaper cover of the present invention, it is also preferred that both of the right waist part and the left waist part are provided with the second attachments, the right waist part has an opening which the left waist part is insertable through, disposed medial in the width direction to the second attachment of the right waist part, and/or the left waist part has an opening which the right waist part is insertable through, disposed medial in the width direction to the second attachment of the left waist part. When the right waist part and the left waist part are constituted in such manners, the right waist part and the left waist part can be fixed around the waist of the wearer while both of the right waist part and the left waist part are pulled. Therefore, fitting property of the diaper cover to the wearer is easily enhanced by the right and left waist parts, and the pelvis is easily supported by the right and left waist parts.

The present invention also provides a method of wearing the diaper cover of the present invention comprising the step of attaching the first attachment to a position on a dorsal side of the wearer in the state where the right flap part and the left flap part are stretched. According to the method of wearing the diaper cover of the present invention, the tension is imparted to the diaper cover from about the groins of the wearer through about the inguinal regions on the abdomen side to the dorsal side of the waist, and as a result, the iliopsoas muscle of the wearer is reinforced by the diaper cover. Therefore, even for elderly people who need to wear a diaper, weakening of legs and back is suppressed by wearing the diaper cover in accordance with the method of wearing the diaper cover of the present invention.

### Advantageous Effects of Invention

The diaper cover of the present invention can support an iliopsoas muscle of a wearer.

### BRIEF DESCRIPTION OF DRAWINGS

FIG. 1 shows one embodiment of a diaper cover of the present invention.

FIG. 2A shows an arrangement example of a first attachment in the diaper cover of the present invention.

FIG. 2B shows an arrangement example of the first attachment in the diaper cover of the present invention.

FIG. 2C shows an arrangement example of the first attachment in the diaper cover of the present invention.

FIG. 2D shows an arrangement example of the first attachment in the diaper cover of the present invention.

FIG. 2E shows an arrangement example of the first attachment in the diaper cover of the present invention.

FIG. 2F shows an arrangement example of the first attachment in the diaper cover of the present invention.

FIG. 3A shows an example of a wearing method of the diaper cover shown in FIG. 1, and shows the diaper cover in the state where the diaper cover is not worn and is spread in a plane.

FIG. 3B shows an example of a wearing method of the diaper cover shown in FIG. 3A, and shows the diaper cover in the state where a right waist part and a left waist part are joined.

FIG. 3C shows an example of a wearing method of the diaper cover shown in FIG. 3B, and shows the diaper cover in the state where the diaper cover is worn.

FIG. 4A shows the diaper cover in the wearing state as viewed from the front side of a wearer.

FIG. 4B shows the diaper cover in the wearing state as viewed from the back side of a wearer.

FIG. 5 shows another embodiment of a diaper cover of the present invention.

FIG. 6A shows an example of a wearing method of the diaper cover shown in FIG. 5, and shows the diaper cover in the state where the diaper cover is not worn and is spread in a plane.

FIG. 6B shows an example of a wearing method of the diaper cover shown in FIG. 6A, and shows the diaper cover in the state where a right waist part and a left waist part are joined.

FIG. 6C shows an example of a wearing method of the diaper cover shown in FIG. 6B, and shows the diaper cover in the state where the diaper cover is worn.

FIG. 7 shows another embodiment of a diaper cover of the present invention.

FIG. 8A shows an example of a wearing method of the diaper cover shown in FIG. 7, and shows the diaper cover in the state where the diaper cover is not worn and is spread in a plane.

FIG. 8B shows an example of a wearing method of the diaper cover shown in FIG. 8A, and shows the diaper cover in the state where a right waist part and a left waist part are joined.

FIG. 8C shows an example of a wearing method of the diaper cover shown in FIG. 8B, and shows the diaper cover in the state where the diaper cover is worn.

### DESCRIPTION OF EMBODIMENTS

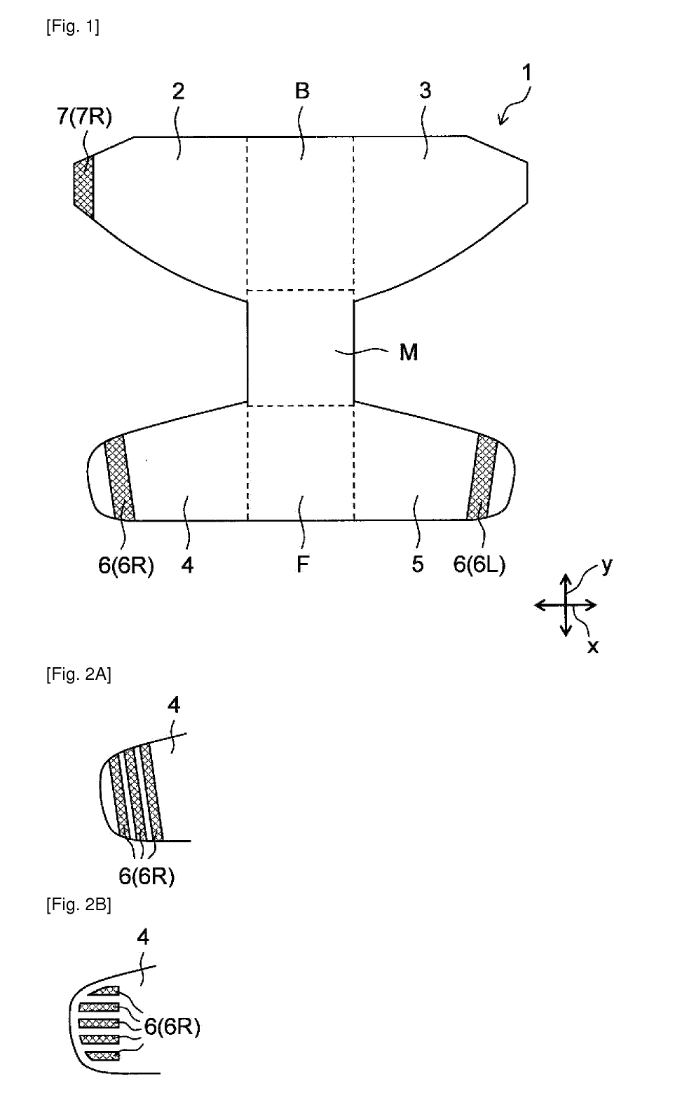

A diaper cover of the present invention is explained referring to drawings. However, the diaper cover of the present invention is not restricted to embodiments shown in the drawings. FIG. 1 shows an example of the diaper cover of the present invention. In the drawings, the arrow x represents a width direction, and the arrow y represents a front-back direction.

A diaper cover 1 has a front-back direction y and a width direction x. The front-back direction y means a direction extending in a front-back direction at a crotch of a wearer when the wearer wears the diaper cover. In the FIG. 1, a lower side of the drawing corresponds to a front side of the diaper cover, and an upper side of the drawing corresponds to a back side of the diaper cover. The width direction x means a direction orthogonal to the front-back direction y on the same plane as the diaper cover. The width direction x corresponds to a direction extending in a right-left direction of a wearer when the wearer wears the diaper cover. The diaper cover 1 also has an inner side located on a wearer's side and an outer side located on opposite side of the wearer when the wearer wears the diaper cover.

The diaper cover 1 comprises a front part F, a back part B and an intermediate part M located therebetween in the front-back direction y. The front part F is formed at one end of the diaper cover 1 in the front-back direction y, and means a portion applied to an abdomen side of a wearer when the diaper cover is worn. The back part B is formed at the other end of the diaper cover 1 in the front-back direction y, and means a portion applied to a dorsal side of the wearer when the diaper cover is worn. The intermediate part M is positioned between the front part F and the back part B, and means a portion applied to a crotch of the wearer when the diaper cover is worn. In the present invention, it is defined that the front part F, the back part B and the intermediate part M are parts excluding a right waist part 2, a left waist part 3, a right flap part 4 and a left flap part 5 described below. In FIG. 1, the front part F, the intermediate part M, the back part B, the right waist part 2, the left waist part 3, the right flap part 4 and the left flap part 5 are shown so as to be sectioned each other by dashed lines, conveniently.

For example, the front part F, the intermediate part M, and the back part B occupy portions equivalent to 30%, 30%, and 40%, respectively, of the length of the diaper cover 1 in the front-back direction y. The diaper cover 1 shown in FIG. 1 is divided into the front part F, the intermediate part M, and the back part B in such a ratio.

The diaper cover 1 comprises the right waist part 2 and the left waist part 3 extending from the back part B in the width direction x. The right waist part 2 and the left waist part 3 are portions applied to a right side and a left side, respectively, of the waist of a wearer when the diaper cover is worn. In FIG. 1, a right side of the drawing corresponds to a left side of a wearer and a left side of the drawing corresponds to a right side of the wearer, when the diaper cover is worn. Thus, the diaper cover of FIG. 1 is shown such that the inner side of the diaper cover is seen. The diaper cover 1 covers the waist of a wearer with the right waist part 2, the left waist part 3 and the back part B when the diaper cover 1 is worn. It is preferred that the diaper cover 1 is worn such that the length of a portion where the right waist part 2 and the left waist part 3 overlap each other in the width direction x is about 10% or more and about 60% or less (more preferably about 20% or more and about 50% or less) of the waist circumference length of a wearer.

The diaper cover 1 is formed such that the intermediate part M is the narrowest in the width direction x. The right waist part 2 and the left waist part 3 correspond to portions located outward from outer edges of the narrowest part of the intermediate part M in the width direction x. The right waist part 2 and the left waist part 3 extend at least from the back part B in the width direction x, and may extend further from the intermediate part M in the width direction x.

The diaper cover 1 comprises the right flap part 4 and the left flap part 5 extending from the front part F in the width direction x. The right flap part 4 and the left flap part 5 are portions which cover the abdomen of a wearer with the front part F when the diaper cover is worn. The right flap part 4 and the left flap part 5 reach to the dorsal side of a wearer when the diaper cover is worn. The right flap part 4 and the left flap part 5 correspond to portions located outward from the outer edges of the narrowest part of the intermediate part M in the width direction x. The right flap part 4 and the left flap part 5 extend at least from the front part F in the width direction x, and may extend further from the intermediate part M in the width direction x.

Each of the right flap part 4 and the left flap part 5 is provided with a first attachment 6. Hereinafter, a reference sign “6R” may be used for representing the first attachment provided on the right flap part, and a reference sign “6L” may be used for representing the first attachment provided on the left flap part. When the diaper cover 1 is worn, the right flap part 4 and the left flap part 5 are joined to the right waist part 2, the left waist part 3 or the back part B which are applied around the waist of a wearer with the first attachment 6.

The first attachments 6 of the right and left flap parts 4, 5 are preferably provided so as to be attachable to the outer surface (the surface located on the outer side) of the right waist part 2, the left waist part 3 or the back part B. For example, the first attachment may be provided on the inner surface (the surface located on the inner side) of the right flap part 4 or the left flap part 5, or may be provided so as to extend from an end, with respect to the width direction x, of the right flap part 4 or the left flap part 5. For improving handleability of the right and left flap parts 4, 5, the first attachments are preferably provided on the inner surface of the right flap part 4 and the inner surface of the left flap part 5, respectively, as shown in FIG. 1.

In the case that the first attachment 6 is provided on the inner surface of each of the right flap part 4 and the left flap part 5, the first attachment 6 is preferably provided within 100 mm (more preferably within 80 mm, and even more preferably within 50 mm) from the end of the right flap part 4 or the left flap part 5 in the width direction x. When the first attachments 6 are provided in such a manner, the first attachments 6 can be attached to the right waist part 2, the left waist part 3 or the back part B while holding the ends of the right flap part 4 and the left flap part 5 in the width direction x, whereby the diaper cover 1 is easily worn. In addition, since the right flap part 4 and the left flap part 5 are easily pulled in the right-left direction of a wearer while holding the ends, with respect to the width direction x, of the right flap part 4 and the left flap part 5, the fitting property of the diaper cover 1 to the wearer is easily enhanced in wearing the diaper cover 1.

Examples of the first attachment include a hook member and a loop member of a hook-and-loop fastener, an adhesive (e.g., an adhesive tape and an adhesive layer), a hook (e.g., a metallic hook and a plastic hook), and the like. In the case that a hook fastener, namely, a hook member of a hook-and-loop fastener, is used as the first attachment, it is preferred that a loop member of the hook-and-loop fastener is provided on the right and left waist part and/or the back part, or the right and left waist part and/or the back part is composed of a material which functions as the loop member, such as a nonwoven fabric, a woven fabric, a knitted fabric or the like. In the case that an adhesive is used as the first attachment, it is preferred that a plastic film is provided on the right and left waist part and/or the back part, or the right and left waist part and/or the back part is composed of a plastic film. In the case that a hook is used as the first attachment, it is preferred that a loop-shaped member which can engage with the hook is provided on the right and left waist part and/or the back part, or a slit which can engage with the hook is formed in the right and left waist part and/or the back part. In this case, it is preferred that a plurality of loop-shape members or slits are provided so that engaging positions of the hook are selectable in accordance with the body size of a wearer. Among them, the hook fastener is preferably used as the first attachment. As the hook fastener, a member having hooks of, for example, an anchor shape, a hook shape, a mushroom shape, or the like may be employed.

A shape of the first attachment, that is a shape of a region where the first attachment is provided in the right flap part or the left flap part, is not particularly limited; however, it is preferably long in the front-back direction y. The first attachment having the shape long in the front-back direction y means that the length of the first attachment in the front-back direction y is longer than that in the width direction x. The first attachment does not need to be provided so as to be parallel to the front-back direction y, and may be provided obliquely with respect to the front-back direction y. When the first attachment has the shape long in the front-back direction y, the right and left flap parts can be widely attached, and thus are tend to be stably fixed on the dorsal side of the wearer. Examples of the shape of the first attachment include, for example, a linear shape, a rectangular shape, an elliptical shape, a rectangular shape whose corners are rounded, an irregular shape and the like; and a practically straight line shape having a width is preferable as the shape of the first attachment.

As shown in FIG. 1, it is preferred that the first attachment 6 has a practically straight line shape having a width and is provided such that the practically straight line shape extends forward in the front-back direction y and inward in the width direction x. That is, it is preferred that the first attachment 6 having a practically straight line shape is provided such that its front side in the front-back direction y is located inward in the width direction x. By providing the first attachment 6 in such a manner, when a wearer pulls the right and left flap parts 4, 5 in a direction perpendicular to the direction in which the practically straight line shape of the first attachment 6 extends, the wearer easily pulls the right and left flap parts 4, 5 upward while expanding the right and left flap parts 4, 5 in the right-left direction of the wearer, whereby tension is easily imparted to the diaper cover 1 from the crotch of the wearer through about the inguinal regions on the abdomen side to the dorsal side of the wearer.

Each of the right flap part and the left flap part may be provided with a plurality of first attachments. When the first attachments are provided in such a manner, all joins of the first attachments are less likely to be disjoined at one time, and thus, the right and left flap parts are tend to be stably fixed on the dorsal side of the wearer.

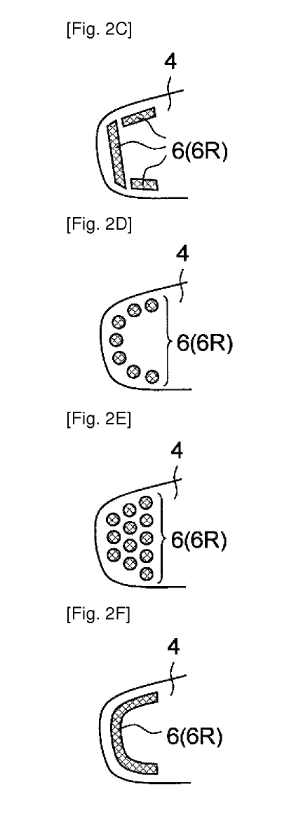

FIGS. 2A to 2E show arrangement examples of the first attachments 6 when the plurality of first attachments 6 are provided, taking the right flap part 4 of the diaper cover 1 shown in FIG. 1 for instance. In FIG. 2A, a plurality of first attachments 6 having shapes long in the front-back direction y are aligned in the width direction x. As shown in FIG. 2B, each first attachment 6 may have a shape long in the width direction x, and these first attachments 6 may be aligned in the front-back direction y. Further, as shown in FIGS. 2C and 2D, a plurality of first attachments 6 may be provided along the edge of the end part of the right flap part 4. Still further, as shown in FIG. 2E, the first attachments 6 having predetermined shapes may be arranged in a matrix in a plane on the end part of the right flap part 4.

In addition, as shown in FIG. 2F, the first attachment may be provided so as to extend arcuately along the edge of the end part of the right flap part or the left flap part. In this case, the edges of the end parts of the right flap part and the left flap part are preferably configured arcuately. By disposing the first attachments in such a shape, the first attachments provided on the end parts of the right flap part and the left flap part are unlikely to be accidentally attached to other members or brought into contact with the skin of the wearer, and hence, handleability of the right and left flap parts improves.

In the diaper cover 1, the right flap part 4 and the left flap part 5 are stretchable, and the first attachment 6 is attachable to a position on the dorsal side of a wearer in the state where the right flap part 4 and the left flap part 5 are stretched. Thus, the right flap part 4 and the left flap part 5 have lengths so as to reach to the dorsal side of a wearer in the stretched state. The right flap part 4 and the left flap part 5 is preferably stretchable at least in the width direction x.

The first attachment 6R of the right flap part 4 is attachable to a position on the right dorsal side of a wearer, and the first attachment 6L of the left flap part 5 is attachable to a position on the left dorsal side of the wearer. The dorsal side of a wearer means a region within less than 25% of the waist circumference length of a wearer from the backbone (specifically, a centerline of the backbone) of the wearer, that is a region less than 50% in all of right and left sides of a wearer. The centerline of the backbone means a centerline extending vertically in the state where the wearer stands.

In the diaper cover 1, the intermediate part M is stretchable in addition to the right flap part 4 and the left flap part 5. When the right flap part 4, the left flap part 5 and the intermediate part M are stretchable, the tension is imparted to the diaper cover 1 from the crotch of the wearer through about the inguinal regions on the abdomen side to the dorsal side of the wearer by fixing the right flap part 4 and the left flap part 5 on the dorsal side of the wearer. The intermediate part M is preferably stretchable at least in the front-back direction y. In addition, the right flap part 4, the left flap part 5 and the intermediate part M are preferably stretchable in a region which surrounds a femoral area of a wearer. Further, the front part F may be also stretchable.

In the diaper cover of the present invention, since the right flap part, the left flap part and the intermediate part are stretchable, and the first attachment is attachable to a position on the dorsal side of a wearer in the state where the right flap part and the left flap part are stretched, the tension is imparted to the diaper cover from about the groins of the wearer through about the inguinal regions on the abdomen side to the dorsal side of the waist. As a result, the iliopsoas muscle of the wearer is reinforced by the diaper cover. The iliopsoas muscle is composed of greater psoas muscle, iliac muscle and the like, and is a group of muscles which connects lumbar spines to the lesser trochanter of a thighbone. That is, the iliopsoas muscle extends from about the groin through a space surrounded by the pelvis to the dorsal side of the waist and the back. The iliopsoas muscle plays an important role in standing posture, walking and running, and if the function of the iliopsoas muscle is depressed, it becomes difficult to make the trunk upright, walk on one's own, and go up and down stairs, for example. In particular, elderly people who need to wear a diaper often have weak legs and back due to a decreased function of the iliopsoas muscle, which interferes with their daily life. However, the diaper cover of the present invention serves so as to keep the pelvis leaned forward and supports the iliopsoas muscle so as to assist the wearer to easily take a standing posture in the state where the wearer stands. When the wearer bends his or her upper body in the front-back direction and the right-left direction of the wearer, the diaper cover acts as a load with respect to the movement of the waist, and thus the iliopsoas muscle is developed. Therefore, even for elderly people who need to wear a diaper, weakening of legs and back is suppressed by wearing the diaper cover of the present invention.

The first attachment is preferably provided so as to be attachable to a position on the dorsal side of the pelvis of a wearer. When the first attachment is fixed to a position on the dorsal side of the pelvis of the wearer in the state where the right flap part and the left flap part are stretched, the portion of the pelvis from about the groins of the wearer to the dorsal side of the waist is certainly supported by the tension of the diaper cover, and thus the wearer easily keeps a standing posture.

It is also preferable that the first attachment is attachable to a position as far as 5% or more and 20% or less of the waist circumference length of a wearer away from the backbone of a wearer. When the first attachment is attached to such a position, the iliopsoas muscle is likely to be certainly supported by the diaper cover. More preferably, the first attachment is attachable to a position as far as 8% or more and 15% or less of the waist circumference length of a wearer away from the backbone of the wearer.

In the diaper cover of the present invention, stretchable parts such as the right flap part, the left flap part and the intermediate part are not particularly limited, as long as the stretchable parts deform with respect to a tensile load and generate forces to return from their deformed shapes to their original shapes. The stretchable parts preferably return substantially to their original lengths even when being stretched at least 1.1-fold (more preferably 1.2-fold, even more preferably 1.5-fold), and preferably do not have yield points in the above stretching range.

The stretch rate of each of the right flap part, the left flap part and the intermediate part, which is defined in JIS L 1018 8.14, is preferably 5% or higher, more preferably 10% or higher, and preferably 60% or lower, more preferably 50% or lower. When the stretch rate of each of the right flap part, the left flap part and the intermediate part is in the range of 5% to 60%, handleability of the diaper cover in wearing improves, and the portion of the wearer from about the inguinal regions to the dorsal side of the waist is easily reinforced by the right flap part, the left flap part and the intermediate part.

The stretch rate means a stretch rate when a constant load is applied, and is measured by the cut strip method. The stretch rate is measured by pulling a test piece having a width of 2.5 cm and a length of 20 cm with a tensile strength tester. Specifically, the test piece is held by pinching tools located in the up-down direction of the tensile strength tester, such that the pinched interval (L₀), that is, the interval between the pinching tools, is 10 cm, and then, a load of 22.1 N (2.25 kgf) is applied to the piece for one minute. The pinched interval (L₁) of the test piece after one minute is measured. A stretch rate Ep (%) is calculated by the following equation: Ep=(L₁−L₀)/L₀×100.

In the diaper cover, it is preferable that the stretch rates of the right flap part and the left flap part are higher than that of the intermediate part. When the stretch rates of the right flap part and the left flap part are higher than that of the intermediate part, the tension of the diaper cover becomes higher about the inguinal regions of the wearer than at the crotch of the wearer. Therefore, the wearing feel of the diaper cover at the crotch improves, and the diaper cover also can effectively support the wearer from about the inguinal regions to the dorsal side of the waist. Furthermore, when the diaper cover is worn, a diaper such as an incontinent pad is easily disposed to the intermediate part without being twisted or bent.

The stretch rate of each of the right flap part, the left flap part, and the intermediate part in the front-back direction y is preferably higher than that in the width direction x. When the stretch rate of each the right flap part, the left flap part, and the intermediate part in the front-back direction y is higher than that in the width direction x, the wearer is easily supported by the diaper cover at a portion from about the inguinal regions to the dorsal side of the waist, more effectively.

An example of a preferable range of the stretch rate of each of the right flap part, the left flap part and the intermediate part in each of the width direction x and the front-back direction y is indicated as follows: the stretch rate of each of the right flap part and the left flap part in the width direction x is preferably in the range of 5% to 30%, and the stretch rate of each of the right flap part and the left flap part in the front-back direction y is preferably in the range of 15% to 30%.

One or both of the right waist part 2 and the left waist part 3 is preferably provided with a second attachment 7. Hereinafter, a reference sign “7R” may be used for representing the second attachment provided on the right waist part, and a reference sign “7L” may be used for representing the second attachment provided on the left waist part. The diaper cover 1 is preferably formed such that the right waist part 2 and the left waist part 3 are joined together with the second attachment 7 when the diaper cover is worn, whereby the right waist part 2 and the left waist part 3 are fixed around the waist of a wearer when the diaper is worn. FIG. 1 shows the embodiment in which only the second attachment 7R is provided in the right waist part 2. In FIG. 1, the right waist part 2 and the left waist part 3 are fixed around the waist of a wearer by joining the right waist part 2 to the left waist part 3 or the back part B with the second attachment 7R.

The second attachment 7R of the right waist part 2 is preferably provided so as to be attachable to the outer surface of the left waist part 3 or the back part B. Also, the second attachment 7L of the left waist part 3 is preferably provided so as to be attachable to the outer surface of the right waist part 2 or the back part B. For example, the second attachment may be provided on the inner surface of the right waist part 2 or the left waist part 3, or may be provided so as to extend from an end, with respect to the width direction x, of the right waist part 2 or the left waist part 3. For improving handleability of the right and left waist parts, the second attachment is preferably provided on the inner surface of the right waist part and/or the inner surface of the left waist part.

In the case that the second attachment 7 is provided on the inner surface of the right waist part 2 or the left waist part 3, the second attachment 7 is preferably provided within 100 mm (more preferably within 80 mm, and even more preferably 50 mm) from the end of the right waist part 2 or the left waist part 3 in the width direction x. When the second attachment 7 is provided in such a manner, the second attachment 7 can be attached to the right waist part 2, the left waist part 3 or the back part B while holding the ends of the right waist part 2 and the left waist part 3 in the width direction x, whereby the diaper cover 1 is easily worn.

As the second attachment, a member which can be used for the first attachment may be used, and the hook fastener is preferably used. As a member which the second attachment joins to and is provided at the right waist part 2, the left waist part 3 or the back part B, and as a material which constitutes the right waist part 2, the left waist part 3 or the back part B, a member or a material which can be used for the member or the material the first attachment joins to may be used.

A shape of the second attachment, that is a shape of a region where the second attachment is provided in the right waist part 2 or the left waist part 3, is not particularly limited; however, it is preferably long in the front-back direction y. The second attachment having the shape long in the front-back direction y means that the length of the second attachment in the front-back direction y is longer than that in the width direction x. The second attachment does not need to be provided so as to be parallel to the front-back direction y, and may be provided obliquely with respect to the front-back direction y. When the second attachment has the shape long in the front-back direction y, the right and left waist parts can be widely attached, and thus are tend to be stably fixed around the waist of the wearer. Examples of the shape of the second attachment include, for example, a linear shape, a rectangular shape, an elliptical shape, a rectangular shape whose corners are rounded, an irregular shape and the like; and a practically straight line shape having a width is preferable as the shape of the second attachment.

The right waist part and/or the left waist part may be respectively provided with a plurality of second attachments. When the second attachments are provided in such a manner, all joins of the second attachments are less likely to be disjoined at one time, and thus, the right and left waist parts are tend to be stably fixed around the waist of the wearer. Examples of the embodiment in which the plurality of second attachments are provided in the right waist part and/or the left waist part are the same as the arrangement examples of the first attachments shown in FIGS. 2A to 2E. Further, as shown in FIG. 2F, the second attachment may be provided so as to extend arcuately along the edge of the end part of the right waist part or the left waist part.

In the diaper cover, the right waist part and the left waist part are preferably stretchable, and the diaper cover is preferably formed such that the right waist part and the left waist part cover iliac crests of a wearer and are joined together with the second attachment when the diaper cover is worn. Thus, when the diaper cover is worn, the right waist part and the left waist part are preferably fixed around the waist of the wearer such that the right waist part and the left waist part cover the iliac crests of the wearer. As a result, both sides of the pelvis are covered with the right waist part and the left waist part to support the pelvis, and thus widening or distortion of the pelvis, which is a cause of low back pain, is easily corrected or prevented by the diaper cover.

Each of the right waist part and the left waist part preferably has the stretch rate comparable to the right flap part and the left flap part. Thus, the stretch rate of each of the right waist part and the left waist part, which is defined in JIS L 1018 8.14, is preferably 5% or higher, more preferably 10% or higher, and preferably 60% or lower, more preferably 50% or lower. When the stretch rate of each of the right waist part and the left waist part is in the range of 5% to 60%, handleability of the diaper cover in wearing improves, and the effect of supporting the pelvis by the right waist part and the left waist part is easily exerted. The detail of measuring method of the stretch rate is explained in the above. More preferably, the stretch rate of each of the right waist part and the left waist part in the width direction x is in the range of 35% to 50%, and the stretch rate of each of the right waist part and the left waist part in the front-back direction y is in the range of 15% to 30%.

The back part may be either stretchable or non-stretchable; however, the stretch rates of the right waist part and the left waist part are preferably higher than that of the back part. In detail, the stretch rates of the right waist part and the left waist part in the width direction x are preferably higher than that of the back part in the width direction x. It is preferred that the right flap part and the left flap part are joined to the back part or its vicinity when the diaper cover is worn, and therefore, when the stretch rate of the back part is lower than that of each of the right waist part and the left waist part, the first attachments of the right and left flap parts can be easily fixed to desired positions on the dorsal side of the wearer by attaching the first attachments to the back part or its vicinity. As a result, the iliopsoas muscle of the wearer is likely to be surely reinforced by the diaper cover.

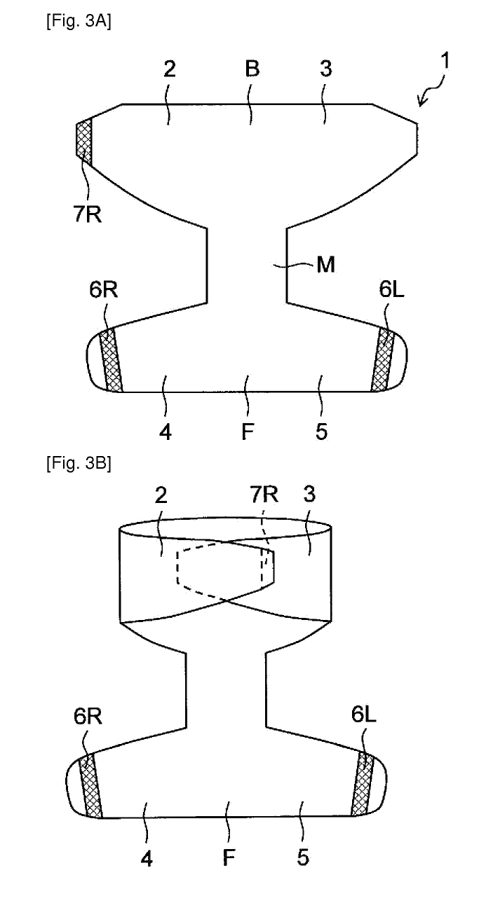

An example of a wearing method of the diaper cover shown in FIG. 1 is explained referring to FIGS. 3A to 3C. In the diaper cover 1 shown in FIGS. 3A to 3C, the second attachment 7 is provided only on the right waist part 2, and the right waist part 2, the left waist part 3 and the back part B are composed of a material to which the first attachment 6 and the second attachment 7 are attachable.

For wearing the diaper cover 1, firstly, while the back part B is in contact with the dorsal side of a wearer, the right waist part 2 is brought into contact with the right side of the waist of the wearer, and the left waist part 3 is brought into contact with the left side of the waist of the wearer. Secondly, the second attachment 7R of the right waist part 2 is attached to the outer surface of the left waist part 3 while pulling the right waist part 2 (see FIGS. 3A and 3B). As a result, the right and the left waist parts 2, 3 are fixed around the waist of the wearer. Thirdly, the front part F, the right flap part 4 and the left flap part 5 are brought through the crotch of the wearer to the abdomen side of the wearer; the right flap part 4 and the left flap part 5 are pulled in the upward direction of the wearer while being pulled in the right-left direction of the wearer; and the right flap part 4 and the left flap part 5 are attached on the dorsal side of the wearer with the first attachments 6R, 6L (see FIGS. 3B and 3C). As a result, the diaper cover 1 can be worn.

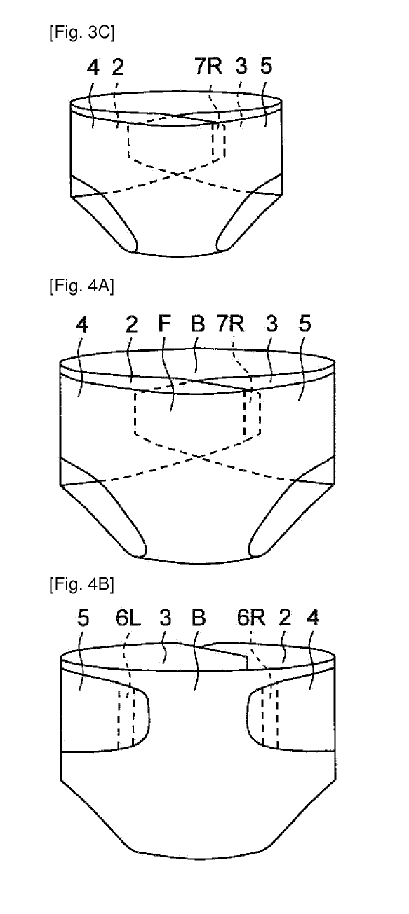

FIGS. 4A and 4B show shapes of the diaper cover, shown in FIG. 1, during wearing. FIG. 4A shows the diaper cover as viewed from the front side of a wearer, and FIG. 4B shows the diaper cover as viewed from the back side of a wearer. In the diaper cover 1, the first attachments 6R, 6L are attached to a position on the dorsal side of the wearer in the state where the right flap part 4 and the left flap part 5 are stretched, as shown in FIG. 4B. Therefore, when the diaper cover 1 is worn, the front side of the wearer appears to be covered with one piece of fabric which is composed of the right flap part 4, the left flap part 5 and the front part F, as shown in FIG. 4A. Thus, the diaper cover 1 looks like shorts as viewed from the front side of the wearer and has a good appearance when being worn.

In order that the right waist part and the left waist part easily cover the iliac crests of a wearer and support the pelvis, the diaper cover preferably has a shape such that the right waist part and the left waist part can be fixed around the waist of the wearer while both of the right waist part and the left waist part are pulled. For example, the diaper cover may be configured as follows: both of the right waist part and the left waist part are provided with the second attachments, and the end of one of the right waist part and the left waist part is located anterior to the end of the other of the right waist part and the left waist part in the front-back direction, whereby the right waist part and the left waist part are joinable together so as to be displaced each other in the front-back direction. Alternatively, the diaper cover may be configured as follows: both of the right waist part and the left waist part are provided with the second attachments, the right waist part has an opening which the left waist part is insertable through, disposed medial in the width direction to the second attachment of the right waist part, and/or the left waist part has an opening which the right waist part is insertable through, disposed medial in the width direction to the second attachment of the left waist part. These embodiments are explained in the following. In the following, the description of parts overlapping the embodiment shown in FIGS. 1 to 3C are omitted.

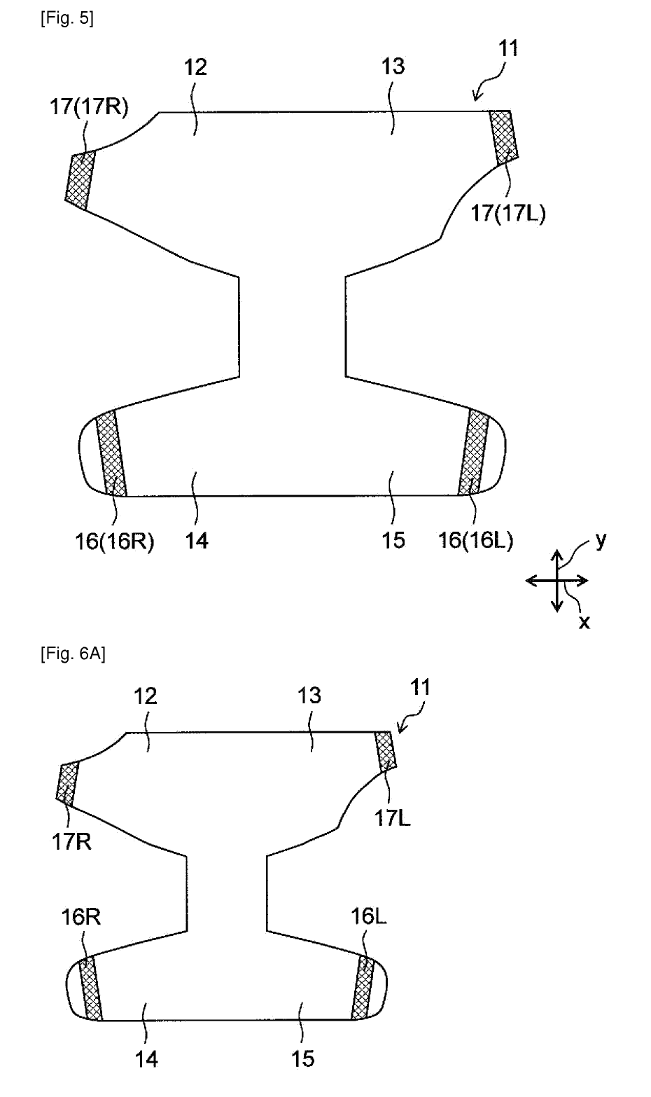

FIG. 5 shows a diaper cover in which both of the right waist part and the left waist part are provided with the second attachments, and the end of one of the right waist part and the left waist part is located anterior to the end of the other of the right waist part and the left waist part in the front-back direction, whereby the right waist part and the left waist part are joinable together so as to be displaced each other in the front-back direction.

In a diaper cover 11 shown in FIG. 5, the end of one of a right waist part 12 and a left waist part 13 is located anterior to the end of the other of the right waist part 12 and the left waist part 13 in the front-back direction y. In FIG. 5, the end of the right waist part 12 is located anterior to the end of the left waist part 13. Therefore, the diaper cover 11 is formed such that the right waist part 12 and the left waist part 13 are joinable together so as to be displaced each other in the front-back direction y. As a result, in wearing the diaper cover 11, a wearer can pull both of the right waist part 12 and the left waist part 13 in the right-left direction of the wearer while holding the ends of the right waist part 12 and the left waist part 13. Therefore, fitting property of the diaper cover 11 around the waist of the wearer is easily enhanced by the right and left waist parts 12, 13, and the pelvis is easily supported by the right and left waist parts 12, 13.

When the diaper cover 11 is worn, the right waist part 12, the left waist part 13 and the back part cover the waist circumference of the wearer, and the right waist part 12 and the left waist part 13 are joined together so as to be displaced each other in the front-back direction y on the abdomen side of the wearer. In the state where the wearer stands, the right waist part 12 and the left waist part 13 are joined together so as to be vertically displaced each other.

Widths of the right waist part 12 and the left waist part 13 are preferably decreased toward their ends, in order to facilitate joining of the second attachments 17. Here, the widths of the right waist part 12 and the left waist part 13 mean lengths with respect to the front-back direction y. When the right and left waist parts 12, 13 are configured in such a manner, a second attachment 17R of the right waist part 12 is easily attached to the left waist part 13 at a position located medially in the width direction x, and a second attachment 17L of the left waist part 13 is easily attached to the right waist part 12 at a position located medially in the width direction x. Further, the hip of the wearer is widely covered with the right waist part 12 and the left waist part 13, thereby improving the wearing feeling of the diaper cover 11.

For example, each of the right waist part 12 and the left waist part 13 may has an outer part formed at the end in the width direction x and an inner part formed between the back part and the outer part; and the inner parts may be configured symmetrically with respect to the centerline of the diaper 11 in the width direction x and the outer part may be configured asymmetrically with respect to the centerline of the diaper 11 in the width direction x. Even when the right waist part 12 and the left waist part 13 is formed in such a manner, the second attachment 17R of the right waist part 12 is easily attached to the inner part of the left waist part 13 and the second attachment 17L of the left waist part 13 is easily attached to the inner part of the right waist part 12. A ratio of lengths of the outer part and the inner part of each of the right waist part and the left waist part in the width direction x is preferably 3:7 to 7:3 (outer part : inner part).

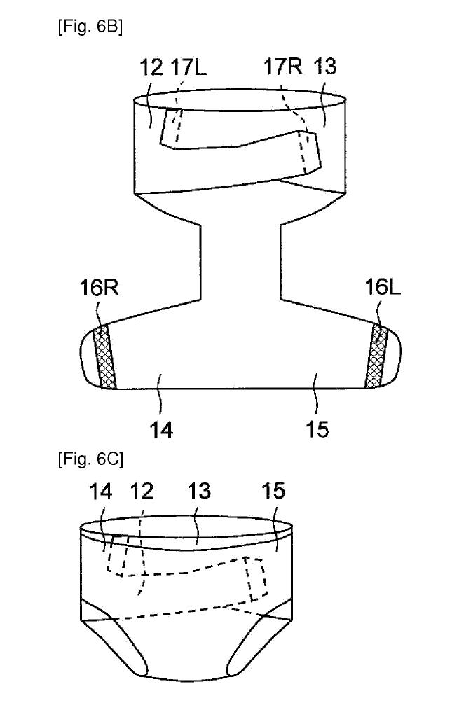

An example of a wearing method of the diaper cover shown in FIG. 5 is explained referring to FIGS. 6A to 6C. In the diaper cover 11 shown in FIGS. 6A to 6C, the right waist part 12, the left waist part 13 and the back part are composed of a material to which the first attachment 16 and the second attachment 17 are attachable. For wearing the diaper cover 11, firstly, while the back part is in contact with the dorsal side of a wearer, the right waist part 12 is brought into contact with the right side of the waist of the wearer, and the left waist part 13 is brought into contact with the left side of the waist of the wearer. Secondly, the second attachment 17R of the right waist part 12 is attached to the outer surface of the left waist part 13 and the second attachment 17L of the left waist part 13 is attached to the outer surface of the right waist part 12, while both of the right waist part 12 and the left waist part 13 being pulled. On this occasion, the right waist part 12 and the left waist part 13 are joined together so as to be displaced each other in the front-back direction y, thereby being fixed around the waist of the wearer (see FIGS. 6A and 6B). Thirdly, the front part, the right flap part 14 and the left flap part 15 are brought through the crotch of the wearer to the abdomen side of the wearer; the right flap part 14 and the left flap part 15 are pulled in the upward direction of the wearer while being pulled in the right-left direction of the wearer; and the right flap part 14 and the left flap part 15 are attached on the dorsal side of the wearer with first attachments 16R, 16L (see FIGS. 6B and 6C). As a result, the diaper cover 11 can be worn.

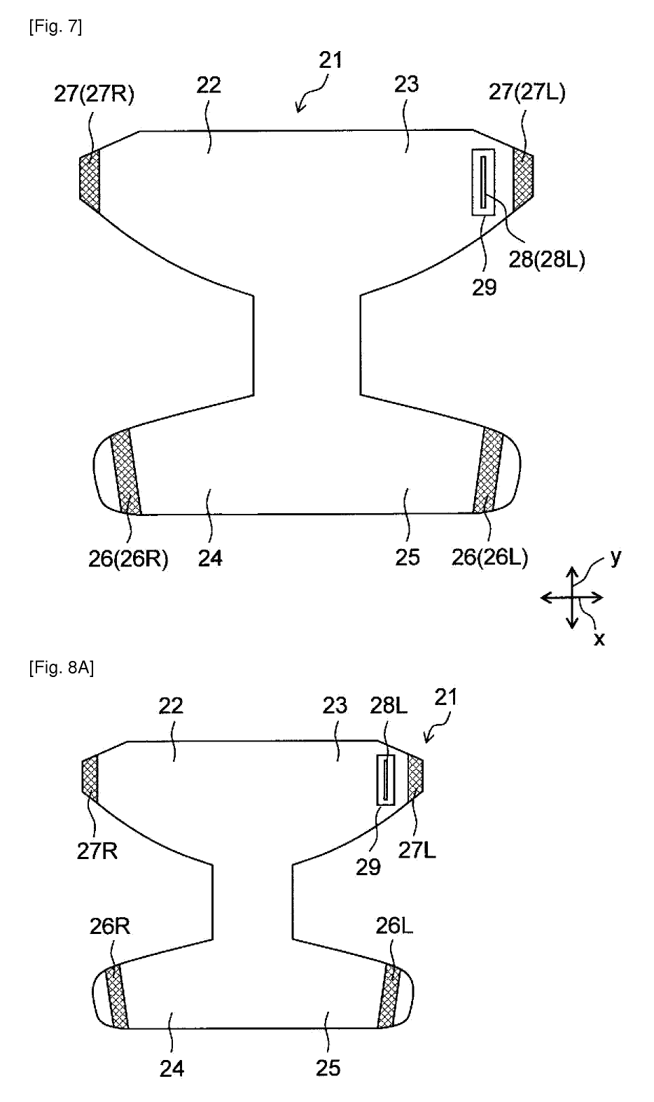

FIG. 7 shows a diaper cover in which both of the right waist part and the left waist part are provided with the second attachments, and the right waist part has an opening which the left waist part is insertable through, disposed medial in the width direction to the second attachment of the right waist part, and/or the left waist part has an opening which the right waist part is insertable through, disposed medial in the width direction to the second attachment of the left waist part.

In a diaper cover 21 shown in FIG. 7, an opening 28L which a right waist part 22 is insertable through is disposed medial in the width direction x to a second attachment 27L of a left waist part 23. In spite of not shown in FIG. 7, in the diaper cover 21, an opening 28R, which is not shown in the drawing, which the left waist part 23 is insertable through may be disposed medial in the width direction x to a second attachment 27R of the right waist part 22. Hereinafter, one or both of the opening 28R and the opening 28L may be simply referred as the opening 28. When the right waist part 22 is provided with the opening 28R, a wearer can pull both of the right waist part 22 and the left waist part 23 by inserting the left waist part 23 through the opening 28R, thereby wearing the diaper cover 21. When the left waist part 23 is provided with the opening 28L, a wearer can pull both of the right waist part 22 and the left waist part 23 by inserting the right waist part 22 through the opening 28L, thereby wearing the diaper cover 21. Therefore, when the diaper cover 21 has the opening 28R and/or the opening 28L, a wearer can pull both of the right waist part 22 and the left waist part 23, thereby wearing the diaper cover 21. As a result, fitting property of the diaper cover 21 to the wearer is easily enhanced by the right and left waist parts 22, 23, and the pelvis is easily supported by the right and left waist parts 22, 23.

The diaper cover 21 is provided with at least one of the opening 28R and the opening 28L, and may be provided with both of the opening 28R and the opening 28L. When the diaper cover 21 is provided with both of the opening 28R and the opening 28L, a wearer can wear the diaper cover 21 by inserting the right waist part 22 or the left waist part 23 through either of the opening 28R or the opening 28L, thereby facilitating wearing of the diaper cover 21.

A shape of the opening 28 is not particularly limited, as long as the right waist part 22 or the left waist part 23 is insertable through the opening 28; however, it is preferably long in the front-back direction y. The opening 28 having the shape long in the front-back direction y means that the length of the opening 28 in the front-back direction y is longer than that in the width direction x. When the opening 28 has the shape long in the front-back direction y, the right waist part 22 or the left waist part 23 can be easily inserted through the opening 28.

Examples of the shape of the opening 28 include, for example, a linear shape, a rectangular shape, an elliptical shape, a rectangular shape whose corners are rounded, an irregular shape and the like. For example, in the case that the opening 28 has a linear shape, a rectangular shape or a elliptical shape, the opening does not need to be provided so as to be parallel to the front-back direction y, and may be provided obliquely with respect to the front-back direction y. As the opening 28, a slit which has a practically straight linear shape long in the front-back direction y is preferred. When the opening 28 is provided in such a shape, narrow portions of the right waist part 22 and the left waist part 23 around the opening 28 are reduced, and therefore, a decrease in the strength becomes small and handleability of the diaper cover improves.

A reinforcement member 29 is preferably provided around the opening 28. By providing the reinforcement member 29 around the opening 28, the diaper cover 21 comes to be hardly torn from the opening 28 as a starting point when the right and left waist parts 22, 23 are pulled in the width direction x. Further, the right waist part 22 or the left waist part 23 comes to be easily inserted through the opening 28.

In order that the diaper cover 21 is hardly torn from the opening 28 as the stating point, the reinforcement member 29 is preferably provided such that its edge closest to the opening 28 is located within 10 mm (more preferably within 5 mm) from the edge of the opening 28. Meanwhile, even when the reinforcement member 29 is provided in a wide area, the suppression effect of tearing of the diaper cover 21 may not be enhanced, and handleabilities of the right and left waist parts 22, 23 may be deteriorated. Therefore, the reinforcement member 29 is preferably provided such that its edge furthest from the opening 28 is located within 50 mm (more preferably within 30 mm) from the edge of the opening 28.

As the reinforcement member 29, a material which is the same as or different from those of the right and left waist parts 22, 23 in which the opening 28 is provided, may be mounted around the opening 28. Alternatively, a mass per unit area of the material constituting the diaper cover 21 may be increased only around the opening 28. Still alternatively, the portion of the right and left waist parts 22, 23 to which the reinforcement member 29 is provided, may be formed from a material having a higher strength than its surrounding. In light of ease of production of the diaper cover 21, as the reinforcement member 29, a material which is the same as or different from those of the right and left waist parts 22, 23 is preferably mounted around the opening 28.

Shapes of the right waist part 22 and the left waist part 23 are not particularly limited; however, a width of the left waist part 23 is preferably decreased toward its end in the case that the right waist part 22 is provided with the opening 28R, and a width of the right waist part 22 is preferably decreased toward its end in the case that the left waist part 23 is provided with the opening 28L. Thus, it is preferred that the right waist part 22 has the opening 28R and the width of the left waist part 23 is decreased toward its end, and/or the left waist part 23 has the opening 28L and the width of the right waist part 22 is decreased toward its end. When the right and left waist parts 22, 23 are configured in such a manner, the right waist part 22 or the left waist part 23 is easily inserted through the opening 28. Here, the widths of the right waist part 22 and the left waist part 23 respectively mean lengths with respect to the front-back direction y.

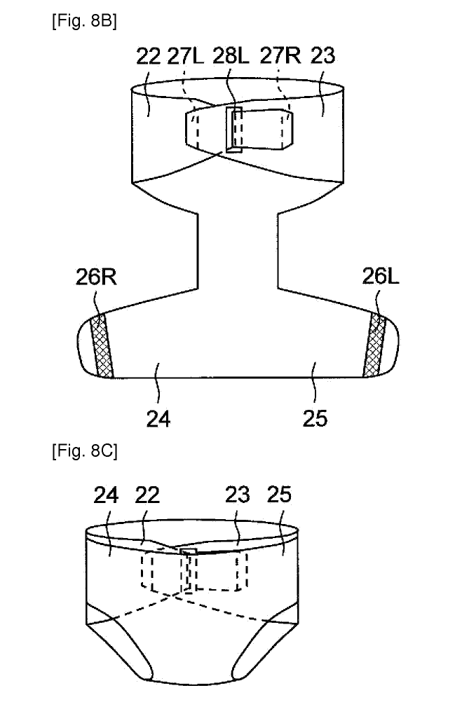

An example of a wearing method of the diaper cover shown in FIG. 7 is explained referring to FIGS. 8A to 8C. In the diaper cover 21 shown in FIGS. 8A to 8C, the opening 28 is formed only in the left waist part 23, and the right waist part 22, the left waist part 23 and the back part are composed of a material to which the first attachment 26 and the second attachment 27 are attachable.

For wearing the diaper cover 21, firstly, while the back part is in contact with the dorsal side of a wearer, the right waist part 22 is brought into contact with the right side of the waist of the wearer, and the left waist part 23 is brought into contact with the left side of the waist of the wearer. Secondly, the right waist part 22 is inserted through the opening 28L of the left waist part 23, and the second attachment 27R of the right waist part 22 is attached to the outer surface of the left waist part 23 and the second attachment 27L of the left waist part 23 is attached to the outer surface of the right waist part 22, while pulling both of the right waist part 22 and the left waist part 23 (see FIGS. 8A and 8B). As a result, the right and left waist parts 22, 23 are fixed around the waist of the wearer. Thirdly, the front part, the right flap part 24 and the left flap part 25 are brought through the crotch of the wearer to the abdomen side of the wearer; the right flap part 24 and the left flap part 25 are pulled in the upward direction of the wearer while being pulled in the right-left direction of the wearer; and the right flap part 24 and the left flap part 25 are attached on the dorsal side of the wearer with first attachments 26R, 26L (see FIGS. 8B and 8C). As a result, the diaper cover 21 can be worn.

A material constituting the diaper cover is not particularly limited. Examples of the material constituting the diaper cover include a nonwoven fabric, a woven fabric, a knitted fabric, a plastic film, a laminate thereof, and the like. In the case that fabrics such as a nonwoven fabric, a woven fabric or a knitted fabric are used as the material constituting the diaper cover, synthetic fibers such as polypropylene, polyethylene, polyesters, polyamides and polyurethanes; or natural fibers such as pulp and silk may be used as fibers constituting the fabric, for example. In the case that a plastic film is used as the material constituting the diaper cover, synthetic resins such as polypropylene, polyethylene, polyesters, polyamides and polyurethanes may be used. The diaper cover may be either liquid-permeable or liquid-impermeable.

A part of the diaper cover which is stretchable, that may be hereinafter referred as “a stretchable part”, may be composed of a stretchable material. Examples of the stretchable material include natural rubbers; synthetic rubbers such as styrene-butadiene copolymer and polyisobutylene; synthetic resins such as polyurethanes, polyether-polyester, polybutylene terephthalate and polytrimethylene terephthalate; and the like. A nonwoven fabric, a woven fabric, a knitted fabric or the like which are composed of fibers containing the stretchable material may be used for the stretchable part. Alternatively, a film which the stretchable material is formed into may be used for the stretchable part.

The stretchable part may be composed of a non-stretchable material. In this case, a nonwoven fabric, a woven fabric or the like which are composed of fibers formed from the non-stretchable material and is imparted stretching property by a weaving or knitting method may be used for the stretchable part.

The stretchable part may be composed of the stretchable material and the non-stretchable material. In this case, to a part which is non-stretchable and is composed of a non-stretchable material, that may be hereinafter referred as “a non-stretchable part”, a stretchable material such as an elastic member and the like may be attached. The stretchable material is preferably attached to the non-stretchable part in a stretched state. Further, a nonwoven fabric, a woven fabric, a knitted fabric or the like which are composed of fibers formed from the stretchable material and fibers formed from the non-stretchable material, or which are composed of composite fibers formed from the stretchable material and the non-stretchable material, may be used for the stretchable part.

For ensuring breathability of the diaper cover and lowering moisture therein during wearing, the diaper cover is preferably composed of a nonwoven fabric, a woven fabric, a knitted fabric or the like. More preferably, the entire diaper cover except the first attachment and the second attachment is composed of a nonwoven fabric, a woven fabric, a knitted fabric or the like. In this case, the hook fastener is preferably used as the first attachment and the second attachment.

In the case that the diaper cover has different stretch rates in the width direction and the front-back direction, the diaper cover is preferably composed of a woven fabric. In this case, different yarns for the warp and the weft of the woven fabric may be used, thereby generating a difference in stretch rate between the width direction and the front-back direction. For example, finenesses, materials, or weaving densities may be made to be different between the warp and the weft. As a yarn constituting the woven fabric, a polyurethane yarn is preferably used. Further, in order to improve a skin feel of a woven fabric containing a polyurethane yarn, a composite yarn containing a polyurethane yarn is preferably used as a yarn constituting the woven fabric. For example, a composite yarn of a polyurethane yarn and a natural fiber such as cotton is preferably used, and a core spun yarn in which a polyurethane yarn is coated with a natural fiber such as cotton is more preferably used.

### REFERENCE SIGNS LIST

1, 11, 21: a diaper cover

2, 12, 22: a right waist part

3, 13, 23: a left waist part

4, 14, 24: a right flap part

5, 15, 25: a left flap part

6, 16, 26: a first attachment

7, 17, 27: a second attachment

1. A diaper cover comprising a front part, a back part and an intermediate part located between the front part and the back part in a front-back direction, a right waist part and a left waist part extending from the back part in a width direction, and a right flap part and a left flap part extending from the front part in the width direction, wherein
each of the right flap part and the left flap part is provided with a first attachment,
the right flap part, the left flap part and the intermediate part are stretchable, and
the first attachment is attachable to a position on a dorsal side of a wearer in a state where the right flap part and the left flap part are stretched.

2. The diaper cover according to claim 1, wherein the first attachment is attachable to a position on a dorsal side of a pelvis of the wearer.

3. The diaper cover according to claim 1, wherein
the right waist part and the left waist part are stretchable,
one or both of the right waist part and the left waist part is provided with a second attachment, and
the diaper cover is formed such that the right waist part and the left waist part cover iliac crests of the wearer and are joined together with the second attachment when the diaper cover is worn.

4. The diaper cover according to claim 1, wherein the first attachment is attachable to a position as far as 5% or more and 20% or less of a waist circumference length of the wearer away from a backbone of the wearer.

5. The diaper cover according to claim 1, wherein a stretch rate of each of the right flap part, the left flap part and the intermediate part in the front-back direction is higher than that in the width direction.

6. The diaper cover according to claim 1, wherein the stretch rates of the right flap part and the left flap part are higher than that of the intermediate part.

7. The diaper cover according to claim 1, wherein the stretch rates of the right waist part and the left waist part are higher than that of the back part.

8. The diaper cover according to claim 1, wherein
the first attachment has a practically straight line shape having a width, and
the first attachment is provided such that the practically straight line shape extends forward in the front-back direction and inward in the width direction.

9. The diaper cover according to claim 1, wherein
both of the right waist part and the left waist part are provided with the second attachments,
an end of one of the right waist part and the left waist part is located anterior to an end of the other of the right waist part and the left waist part in the front-back direction, whereby the right waist part and the left waist part are joinable together so as to be displaced each other in the front-back direction.

10. The diaper cover according to claim 1, wherein
both of the right waist part and the left waist part are provided with the second attachments,
the right waist part has an opening which the left waist part is insertable through, disposed medial in the width direction to the second attachment of the right waist part, and/or the left waist part has an opening which the right waist part is insertable through, disposed medial in the width direction to the second attachment of the left waist part.

11. A method of wearing the diaper cover according to claim 1, comprising the step of attaching the first attachment to a position on a dorsal side of the wearer in a state where the right flap part and the left flap part are stretched.
